(19) United States
(12) Patent Application Publication
(10) Pub. No.: US 2025/0124676 A1
(43) Pub. Date: Apr. 17, 2025

(54) SYSTEMS AND METHODS FOR AUGMENTED REALITY SURGICAL ENVIRONMENT

(71) Applicant: Life Spine, Inc., Huntley, IL (US)

(72) Inventor: Garrett LAUF, Hampshire, IL (US)

(21) Appl. No.: 18/916,230

(22) Filed: Oct. 15, 2024

Related U.S. Application Data
(60) Provisional application No. 63/590,550, filed on Oct. 16, 2023.

(51) Int. Cl. G06T 19/00; A61B 34/10; A61B 34/20; A61B 90/00

(52) U.S. Cl. CPC G06T 19/006; A61B 34/10; A61B 34/20; A61B 90/361; A61B 90/37; A61B 2034/107; A61B 2090/365; A61B 2090/376

(57) ABSTRACT

An augmented reality (AR) display system for a surgical environment includes a wearable AR display device, a scope, and processing circuitry. The wearable AR display device is configured to be worn by a surgeon and provide AR imagery to the surgeon. The scope is configured to be inserted into a patient. The processing circuitry is configured to obtain real-time image data from the scope, X-ray image data of the patient, and navigation data for a surgical procedure of the patient. The processing circuitry is also configured to generate an AR environment that includes the real-time image data, the X-ray image data, and the navigation data. The processing circuitry is also configured to operate the wearable AR display device to provide the AR environment including the real-time image data, the X-ray data, and the navigation data as the AR imagery.

### CROSS-REFERENCE TO RELATED PATENT APPLICATION

This application claims the benefit of and priority to U.S. Provisional Application No. 63/590,550, filed Oct. 16, 2023, the entire disclosure of which is incorporated by reference herein.

### BACKGROUND

The present disclosure relates to display systems. More specifically, the present disclosure relates to augmented reality (AR) display systems.

### SUMMARY

One embodiment of the present disclosure is an augmented reality (AR) display system for a surgical environment. The AR display system includes a wearable AR display device, a scope, and processing circuitry. The wearable AR display device is configured to be worn by a surgeon and provide AR imagery to the surgeon. The scope is configured to be inserted into a patient. The processing circuitry is configured to obtain real-time image data from the scope, X-ray image data of the patient, and navigation data for a surgical procedure of the patient. The processing circuitry is also configured to generate an AR environment that includes the real-time image data, the X-ray image data, and the navigation data. The processing circuitry is also configured to operate the wearable AR display device to provide the AR environment including the real-time image data, the X-ray data, and the navigation data as the AR imagery.

Another embodiment of the present disclosure is display system for a surgical environment. The display system includes a scope and processing circuitry. The scope includes a shaft and a camera disposed at an end of the shaft. The shaft including a bend. The end of the shaft is configured to be inserted into a patient. The processing circuitry is configured to obtain real-time image data from a scope that is configured to be inserted into a patient, X-ray image data of the patient from an X-ray database, and navigation data from a navigation database for a surgical procedure of the patient. The processing circuitry is configured to generate an AR environment that includes the real-time image data, the X-ray image data, and the navigation data. The processing circuitry is configured to operate a wearable AR display device to provide the AR environment including the real-time image data, the X-ray data, and the navigation data as the AR imagery.

Another embodiment of the present disclosure is a method of providing augmented reality (AR) for a surgical environment. The method includes providing a scope having a shaft and a camera disposed at an end of the shaft. The shaft includes a bend. The end of the shaft is configured to be inserted into a patient. The method includes obtaining real-time image data from the scope during a surgical procedure, navigation data for the surgical procedure, and X-ray image data of the patient. The method also includes generating an AR environment based on the real-time image data, the navigation data, and the X-ray image data. The AR environment includes a size and position of the real-time image data, the navigation data, and the X-ray image data. The method also includes operating an AR display device to provide the AR environment including the real-time image data, the navigation data, and the X-ray image data.

### BRIEF DESCRIPTION OF THE DRAWINGS

FIG. 1 is a block diagram of an AR display system for a surgical environment, according to some embodiments.

FIG. 2 is a block diagram of a controller of the AR display system of FIG. 1, according to some embodiments.

FIG. 3 is diagram of an AR display device of the AR display system of FIG. 1, according to some embodiments.

FIG. 4 is a diagram illustrating an AR environment provided by the AR display system of FIG. 1, according to some embodiments.

FIG. 5 is a flow diagram of a process for providing an AR environment in a surgical setting, according to some embodiments.

FIG. 6 is a side view of a scope for the AR display system of FIG. 1, according to some embodiments.

FIG. 7 is a perspective view of an end of the scope of FIG. 6, according to some embodiments.

FIG. 8 is a side view of a base portion of the scope of FIG. 6, according to some embodiments.

FIG. 9 is a side view of the end of the scope of FIG. 6, according to some embodiments.

### DETAILED DESCRIPTION

Before turning to the figures, which illustrate the exemplary embodiments in detail, it should be understood that the present disclosure is not limited to the details or methodology set forth in the description or illustrated in the figures. It should also be understood that the terminology used herein is for the purpose of description only and should not be regarded as limiting.

### Overview

Referring generally to the FIGURES, an AR system includes a wearable AR device, and a controller including processing circuitry. The processing circuitry is configured to receive various image inputs such as X-ray imagery, navigation data or imagery, and real-time image data from a scope that is used during a surgical procedure. The processing circuitry is configured to operate the wearable AR device in order to display the various image inputs in an AR manner such that the various image inputs are provided in separate locations. The locations, sizes, and orientations of the image inputs may be adjustable by the user. Advantageously, the AR system facilitates improved efficiency in a surgical setting since the surgeon may view the various image inputs in a hands-free and customizable manner.

### AR Display System

### System Overview

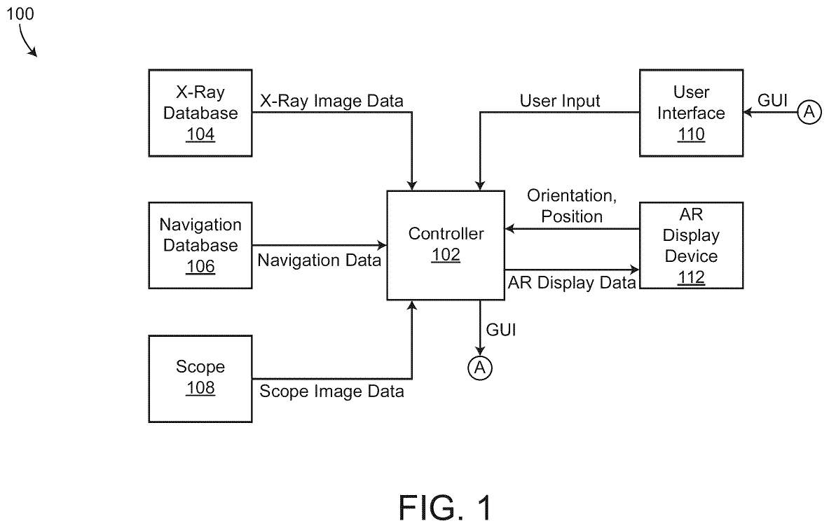

Referring to FIG. 1, an AR display system 100 (e.g., a mixed reality display system, a combiner display system, a waveguide display system, a surgical display system, a control system, etc.) includes a controller 102 (e.g., a processor, a processing unit, multiple processors, a computer, a personal computer device, a handheld computer device, a tablet, a smartphone, etc.), an X-ray database 104 (e.g., a storage or computer readable medium for X-ray image data), a navigation database 106 (e.g., a storage or computer readable medium for navigation data for one or more surgical actions, a set of instructions, guidelines for various surgical procedures and corresponding images or computer generated graphics, etc.), a scope 108 (e.g., a surgical scope including a camera or imaging device configured to be inserted into a patient's body or a patient's spine), a user interface 110 (e.g., a display screen, a touch screen, etc.), and an AR display device 112 (e.g., a wearable device, glasses, a wearable headset, etc.). The controller 102 may be a computer of the AR display device 112 or may communicate with a computer, processor, etc., of the AR display device 112. In some embodiments, the controller 102 includes multiple computers and is a distributed processing system including processing units, processors, etc., of a personal computer device, tablet, or base station, and one or more processors, processing units, etc., of the AR display device 112. It should be understood that any of the processes of the controller 102 as presented herein may be performed at least partially by a processor of the AR display device 112, a cloud computing system, a local processor (e.g., a processor of a tablet, a workstation, a base computer, etc.), or any combination thereof.

The controller 102 is configured to obtain X-ray image data from the X-ray database 104, navigation data from the navigation database 106, and scope image data from the scope 108, according to some embodiments. In some embodiments, the scope image data is live image data (e.g., a video stream) that is obtained from the scope 108 in real-time. In some embodiments, the X-ray database 104 and the navigation database 106 are computer readable medium of a remote computing system, a cloud computing system, a hospital system, etc. In some embodiments, the X-ray database 104 is a database of the controller 102. In some embodiments, the controller 102 may retrieve, from the X-ray database 104, one or more X-rays, provided as X-ray image data, for a patient for which a surgical procedure is to be performed. In some embodiments, the controller 102 provides a query or request to the X-ray database 104 and receives the X-ray image data for the patient from the X-ray database 104 in response to the query or request. The X-ray image data provided to the controller 102 by the X-ray database 104 may include X-ray images of a spine of the patient, an arm, leg, or neck bone of the patient, etc., or more generally, X-ray images of a bone or portion of the patient's body proximate or at the location at which the surgical procedure is to be performed. In other embodiments, the controller 102 is configured to obtain other types or combinations of image, data, and/or other information relating to the surgical procedure being performed.

Referring still to FIG. 1, the navigation data provided to the controller 102 by the navigation database 106 may include procedure-specific data including but not limited to images, instructions, guidelines, computer generated imagery, steps, etc., of the procedure to be performed on the patient. In some embodiments, the navigation data provided by the navigation database 106 includes specific data regarding an implant of the surgical procedure if the procedure is an implant procedure. The navigation data may include manufacturer specific information of the implant, indicating one or more steps or guidances for the implant and the implant procedure. In some embodiments, the navigation data may be displayed sequentially. For example, at different steps of the surgical procedure, different navigation data may be presented to a user of the display system 100 as the user (e.g., the surgeon) performs the surgical procedure.

Referring still to FIG. 1, the scope 108 is configured to provide the scope image data to the controller 102 such that live image data obtained from a camera or imaging device of the scope 108 can be displayed via the AR display device 112. The scope 108 may be a handheld device including an elongated member with an imaging device or camera disposed along the elongated member. The scope 108 may be insertable by the surgeon (e.g., the user of the display system 100) such that the surgeon can obtain real-time image data of a surgical area.

Referring still to FIG. 1, the controller 102 is configured to receive a user input from the user interface 110, according to some embodiments. In some embodiments, the user interface 110 is a display screen or a touch screen at which the surgeon or user can provide one or more inputs in order to adjust a virtual environment that is displayed on the AR display device 112. For example, the AR display device 112 is configured to display the X-ray image data, the navigation data, and the scope image data to the surgeon or user via the AR display device 112 in different virtual locations of the virtual environment. The surgeon or the user may provide a user input via the user interface 110 to adjust the virtual locations of the X-ray image data, the navigation data, or the scope image data in the virtual environment according to preference, to scale or descale the X-ray image data, the navigation data, and the scope image data in the virtual environment according to preference, or to display additional or different data in the virtual environment.

Referring still to FIG. 1, the AR display device 112 is configured to receive AR display data from the controller 102 and operate to display the AR display data to the surgeon or the user in an AR or mixed reality environment, according to some embodiments. In some embodiments, the AR display device 112 is configured to provide sensor data to the controller 102 including orientation and/or position of the AR display device 112. The controller 102 may use the orientation and position of the AR display device 112 in order to adjust the AR display data that is provided to and displayed on the AR display device 112. In some embodiments, the AR display device 112 is configured to display the AR display data to the user or the surgeon while allowing the user or surgeon to view surrounding areas of a surgical environment (e.g., real-world objects, the patient that the procedure is being performed on, etc.). In some embodiments, the controller 102 is configured to adjust the AR display data responsive to the orientation and/or position provided by sensors of the AR display device 112 such that the X-ray image data, the navigation data, and the scope image data are maintained in consistent locations about the virtual environment as the surgeon moves or looks in different directions.

Referring still to FIG. 1, the controller 102 is configured to provide a graphical user interface (GUI) to the user interface 110, according to some embodiments. In some embodiments, the GUI includes one or more options to select or adjust where the X-ray image data, the navigation data, or the scope image data are displayed. For example, the user or the surgeon may select between different predetermined virtual locations and/or sizes for the X-ray image data, the navigation data, and/or the scope image data. The GUI may include one or more dials, buttons, or other inputs such that the surgeon can adjust locations and/or sizes of the X-ray image data, navigation data, and scope image data, in the virtual environment, add new panes for additional display of data, remove panes, etc.

### AR Controller

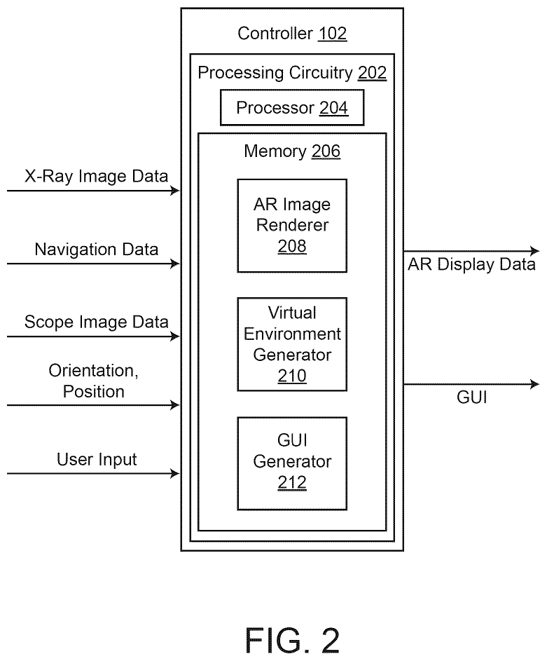

Referring to FIG. 2, the controller 102 of the display system 100 is shown in greater detail, according to some embodiments. In some embodiments, the controller 102 includes processing circuitry 202, a processor 204, and memory 206. Processing circuitry 202 can be communicably connected to a communications interface such that processing circuitry 202 and the various components thereof can send and receive data via the communications interface. Processor 204 can be implemented as a general purpose processor, an application specific integrated circuit (ASIC), one or more field programmable gate arrays (FPGAs), a group of processing components, or other suitable electronic processing components.

Memory 206 (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present application. Memory 206 can be or include volatile memory or non-volatile memory. Memory 206 can include database components, object code components, script components, or any other type of information structure for supporting the various activities and information structures described in the present application. According to some embodiments, memory 206 is communicably connected to processor 204 via processing circuitry 202 and includes computer code for executing (e.g., by processing circuitry 202 and/or processor 204) one or more processes described herein.

In some embodiments, controller 102 is implemented within a single computer (e.g., one server, one housing, etc.). In various other embodiments, controller 102 can be distributed across multiple servers or computers (e.g., that can exist in distributed locations).

The memory 206 includes an AR image renderer 208, a virtual environment generator 210, and a GUI generator 212, according to some embodiments. In some embodiments, the AR image renderer 208 is configured to receive a virtual environment from the virtual environment generator 210, and render AR display data (e.g., image data) according to the virtual environment. For example, the virtual environment may be determined by the virtual environment generator 210 based on the user input(s) provided by the surgeon or the user via the user interface 110. In some embodiments, the virtual environment generator 210 is configured to create the virtual environment based on sizes, positions, and type of data for each of multiple panes. For example, the virtual environment generator 210 may receive, via the user interface 110, a user input to display the scope image data obtained from the scope 108 at a first virtual location or in a first image pane having a first size, a user input to display the navigation data obtained from the scope 108 at a second virtual location or in a second image pane having a second size, and a third user input to display the X-ray image data at a third virtual location or in a third image pane having a third size. In some embodiments, the virtual environment generator 210 is configured to provide the virtual environment to the AR image renderer 208 such that the AR image renderer 208 can operate the AR display device 112 or perform an image rendering technique to output the AR display data.

The AR image renderer 208 is configured to render the AR display data based on the X-ray image data, the navigation data, the scope image data, the virtual environment provided by the virtual environment generator 210, and the orientation/position of the AR display device 112. For example, the AR image renderer 208 may render the AR display data such that the AR display data, when displayed on the AR display device 112, provides the X-ray image data, the navigation data, and the scope image data at the different virtual locations as defined by the virtual environment output by the virtual environment generator 210.

The AR image renderer 208 may update the AR display data, or re-render the AR display data in response to changes in the orientation or position of the AR display device 112 in order to account for the changes in the orientation or position of the AR display device 112 and thereby adjust a field of view that is presented to the surgeon on the AR display device 112 to provide an AR, mixed reality (“MR”), or virtual reality (“VR”) environment to the user via the AR display device 112.

Referring still to FIG. 2, the GUI generator 212 is configured to generate a GUI for the user interface 110, according to some embodiments. The GUI may prompt the user via the user interface 110 to provide an input (e.g., via a slider, a button, etc.) in order to adjust a position or size of one or more display panes, windows, or areas that include the X-ray image data, the navigation data, or the scope image data.

### AR Display Device

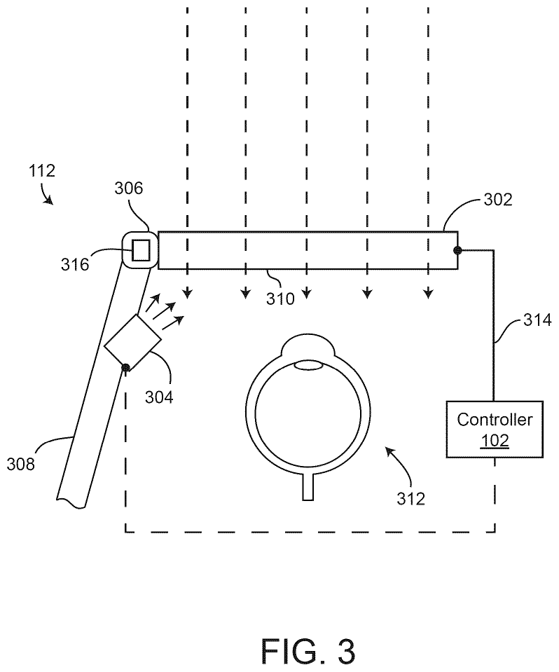

Referring to FIG. 3, the AR display device 112 may include a lens 302 (e.g., a glass surface, a clear member, a transparent member or surface, a translucent member or surface, etc.). In some embodiments, the lens 302 defines a surface 310 that faces the surgeon's eyes 312 and provides a surface onto which imagery (e.g., AR imagery, VR imagery, MR imagery, etc.) is provided. The lens 302 may be supported by one or more structural members, shown as structural member 306 and structural member 308. The structural member 308 may be an arm of a pair of glasses or wearable headset that facilitates wearing of the AR display device 112. For example, the structural member 308 may be coupled with the structural member 306 at a joint positioned at the structural member 306. In some embodiments, the AR display device 112 includes an imaging device 304 (e.g., a projector) that is configured to project AR, VR, or MR imagery onto the surface 310. In some embodiments, the lens 302 is a combiner that is configured to display AR, VR, or MR imagery to the surgeon or the user. The controller 102 may be configured to control or adjust operation of the imaging device 304 or the lens 302 (if the lens is a combiner) in order to provide AR, VR, or MR imagery on the lens 302 to the user or the surgeon. The AR display device 112 also includes one or more orientation or position sensors, shown as orientation and position sensor 316. The orientation and position sensor 316 is configured to provide the orientation and position of the AR display device 112 and therefore the orientation and position of the surgeon or user's head within an environment or space.

### AR Surgical Environment

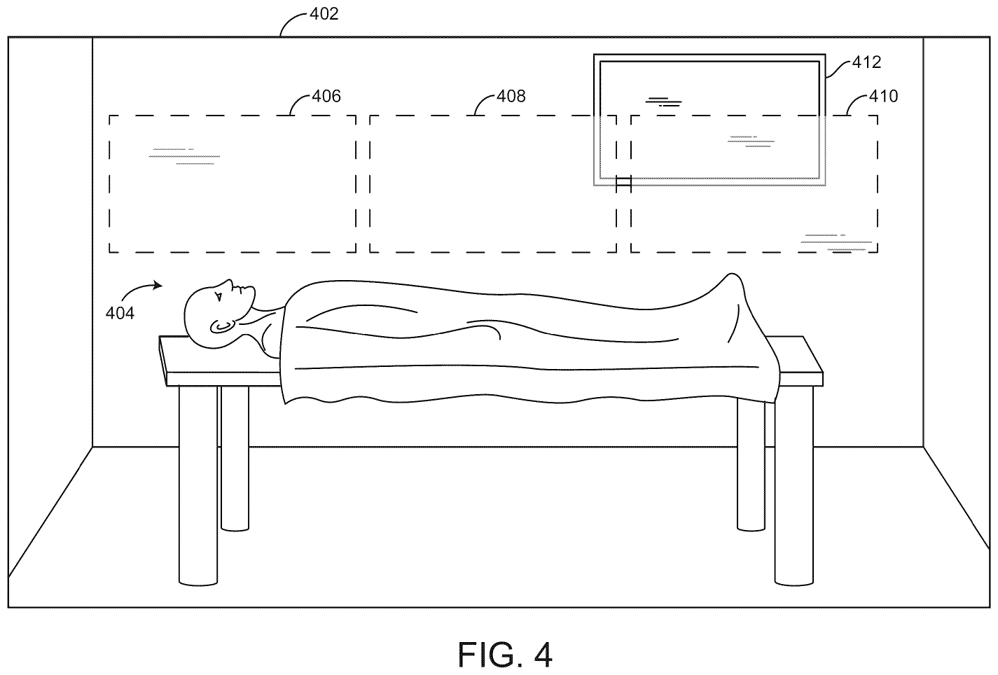

Referring to FIG. 4, a diagram 402 illustrates an AR view or environment that may be provided to the surgeon or the user via the AR display device 112. The diagram 402 illustrates an AR environment which allows the surgeon to view a patient 404 upon which the surgical operation is performed, as well as surroundings (e.g., surrounding medical personnel, room surroundings, etc.). In some embodiments, the AR environment, illustrated by diagram 402, includes AR, VR, or MR imagery including a first pane or window 406, a second pane or window 408, and a third pane or window 410. In some embodiments, the X-ray image data is provided on the first pane or window 406, the scope image data is provided on the second pane or window 408, and the navigation data is provided on the third pane or window 410. In some embodiments, the first pane or window 406 has a corresponding virtual location and size, the second pane or window 408 has a corresponding virtual location and size, and the third pane or window 410 has a corresponding virtual location and size. In some embodiments, the virtual location and size of the window 406, the window 408, and the window 410 are adjustable by the user by providing inputs to the controller 102 via the user interface 110.

Referring still to FIG. 4, the surrounding areas of the real-world environment or surgery room may be viewable through the lens 302. When the surgeon looks around the room (e.g., downwards towards a surgical area, to the left, to the right, etc.), the window 406, the window 408, and the window 410 may maintain their virtual positions and sizes in the AR environment. In some embodiments, the window 406 may be positioned on a left side of the surgery room such that the surgeon can look to the left in order to view the X-ray image data. Similarly, the window 410 may be positioned on a right side of the surgery room such that the surgeon can look to the right in order to view the navigation data. In some embodiments, the window 408 is positioned above the patient 404 such that the surgeon can look up from a surgical site in order to view the scope image data. In other systems, the X-ray image data or the navigation data may be displayed on a physical display screen (e.g., a display screen 412) in the surgery room such that the surgeon must move from the patient 404 or a surgical area to a different part of the room to view the X-ray image data or the navigation data.

The AR environment provided by the display system 100 advantageously allows the surgeon to view the X-ray image data, the navigation data, and the scope image data without requiring the surgeon to move away from the patient 404 in a hands-free manner. For example, the surgeon may view the scope image data in real-time while holding the scope 108 and adjusting position or orientation of the scope. Advantageously, the display system 100 and AR environment provided by the AR display device 112 facilitates improved efficiency during surgical operations.

### Display Process

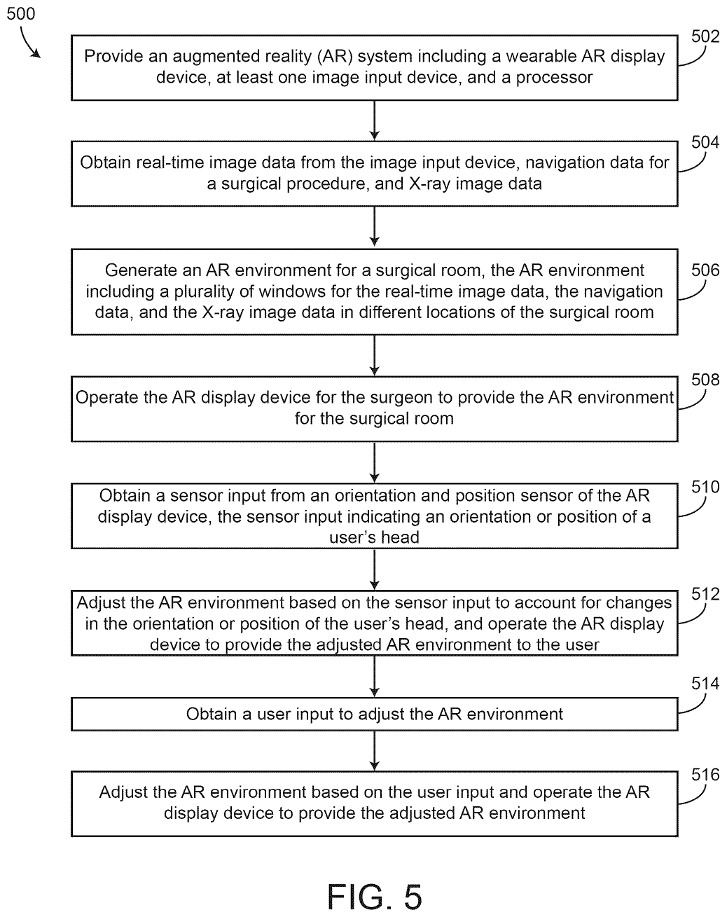

Referring to FIG. 5, a flow diagram of a process 500 for providing an AR environment to a surgeon includes steps 502-516, according to some embodiments. In some embodiments, process 500 is performed in order to display live scope data, navigation data, and X-ray imagery to a surgeon in an AR manner such that the surgeon may view the live scope data, the navigation data, and the X-ray imagery in a hands free manner while conducting a surgery on a patient. Advantageously, providing the live scope data, the navigation data, and the X-ray imagery facilitates improved efficiency of surgical procedures.

The process 500 includes providing an augmented reality (AR) system including a wearable AR display device, at least one image input device, and a processor (step 502), according to some embodiments. In some embodiments, step 502 is performed by providing a packaged system or set of components (e.g., for a surgical procedure) in order to provide an AR surgical environment for the surgeon while performing the surgical procedure. In some embodiments, step 502 includes providing a scope as the image input device, and one or more distributed processors. The wearable AR display device may be a pair of glasses, goggles, etc., including orientation and position sensors, a pair of transparent or translucent lens configured to display AR imagery, a pair of arms configured to support the wearable AR display device on the surgeon's head, etc.

The process 500 includes obtaining real-time image data from the image input device, navigation data for a surgical procedure, and X-ray image data (step 504), according to some embodiments. In some embodiments, the real-time image data is scope data received from an imaging device of a scope that is configured to be inserted into a patient's body (e.g., into the patient's spine) while performing the surgical procedure. In some embodiments, the navigation data is data from a manufacturer of an implant or surgical procedure database including steps for performing the surgical operation, as well as computer generated graphics to guide the surgeon through performing the surgical operation. In some embodiments, the X-ray image data is obtained, by the processor, from an X-ray database. The X-ray image data may include a set of X-rays or images of X-rays of the patient for which the surgical procedure is being performed. In some embodiments, step 504 is performed by the processor of step 502.

The process 500 includes generating an AR environment for a surgical room, the AR environment including multiple windows for display of the real-time image data, the navigation data, and the X-ray image data (step 506), according to some embodiments. In some embodiments, step 506 includes generating a virtual environment including the one or more windows (e.g., flat panes or surfaces provided within the virtual environment at specific locations and with specific orientations). The step 506 may be performed by the processor (e.g., the processing circuitry 202), or more specifically, by the AR image renderer 208 or the virtual environment generator 210.

The process 500 includes operating the AR display device for the surgeon to provide the AR environment for the surgical room (step 508), according to some embodiments. In some embodiments, step 508 includes generating and providing AR display data to AR display device 112 such that the AR display device 112 operates to provide the AR environment to the surgeon in a manner such that the surgeon may view the patient and surrounding environments, and also view the X-ray images data, the navigation data, and the real-time image data obtained from the image input device.

The process 500 includes obtaining a sensor input from an orientation and position sensor of the AR display device, the sensor input indicating at least one of an orientation or position of a user's head (step 510) and adjusting the AR environment based on the sensor input to account for changes in the orientation or position of the user's head and operating the AR display device to provide the adjusted AR environment to the user (step 512), according to some embodiments. In some embodiments, step 510 and step 512 are performed by the AR image renderer 208 and the virtual environment generator 210. The AR environment may be adjusted such that the one or more panes or windows of the data that are displayed to the surgeon (e.g., the X-ray image data, the live image data from the scope, and the navigation data) are maintained in a specific location in the surgeon's field of view, even as the surgeon moves their head to look at different locations in the surgical room.

The process 500 includes obtaining a user input to adjust the AR environment (step 514), according to some embodiments. In some embodiments, step 514 includes providing a graphical user interface (GUI) to the surgeon or user via a display screen. The surgeon or the user may adjust at least one of a location, a size or dimension, or an orientation of any of the windows or panes that display the various data. The GUI may provide sliders or control options for the surgeon or user to adjust the location, size, or orientation of the windows or panes that display the various data. The user input may also be a request to change the position, orientation, or size of any of the windows or panes between predetermined, preset, or stored profiles of different locations, sizes, and orientations of the windows or panes that display the data. In some embodiments, step 514 is performed by the controller 102 by receiving the user input from the user interface 110.

The process 500 includes adjusting the AR environment based on the user input and operating the AR display device to provide the adjusted AR environment (step 516), according to some embodiments. In some embodiments, step 516 is performed similarly to steps 506-508. In some embodiments, step 516 is performed in response to step 514 or at least partially concurrently or simultaneously with step 514 such that the user or surgeon may adjust the positions, orientations, sizes, etc., of the windows or panes upon which imagery is displayed while viewing the adjustments in real-time via the wearable AR display device.

### Scope

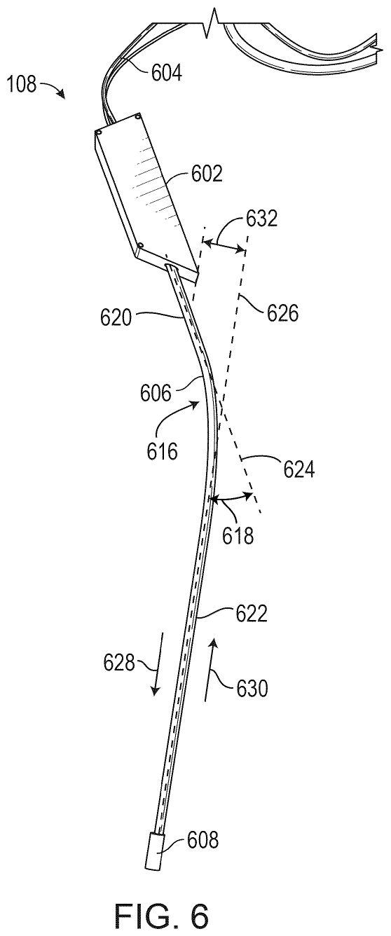

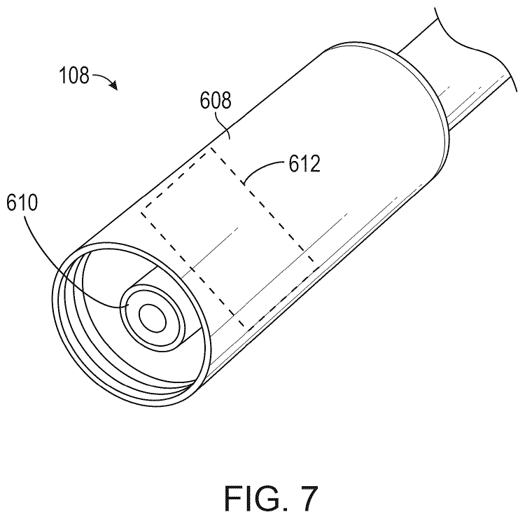

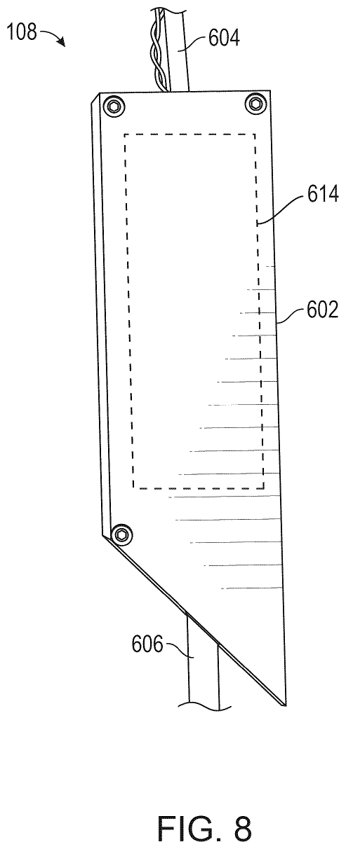

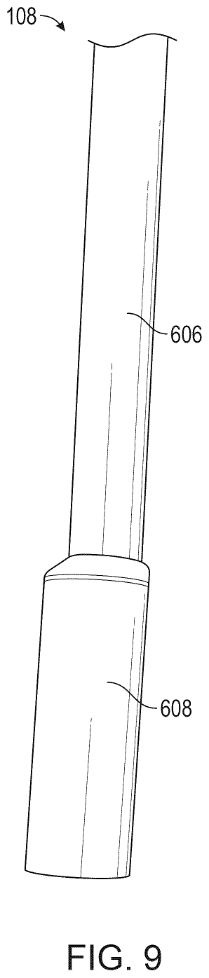

Referring to FIGS. 6-9, the scope 108 includes a base portion 602 (e.g., a housing, a box, a component box, a circuitry box, a controller housing, etc.), a shaft 606 (e.g., an elongated member, an elongated tubular member, etc.), and an end 608 (e.g., a tip, a peripheral end, etc.). The shaft 606 couples with (e.g., is received within, threads into, etc.) a corresponding portion of the base portion 602. The base portion 602 provides a gripping or holding surface for the surgeon to hold the scope 108. The shaft 606 has an elongated shape (e.g., a cylindrical member, an elongated hollow member) that defines a space extending therethrough. One or more wires (e.g., copper wires, electrically conductive wires, fiber optic wires, etc.) extend through the shaft 606 to the end 608. The wires may include an electrical wire for a camera sensor and a light source cable for providing light.

The end 608 may have the form of a shroud that receives a distal end of the shaft 606. The end 608 defines a space within which a camera 610 and a light 612 (e.g., a light emitting diode) are positioned. The camera 610 may wiredly couple with one of the wires that extend through the shaft 606 such that the camera can provide image data. The light 612 wiredly couples with another of the wires that extend through the shaft 606 such that the light can be operated (e.g., via a control device) to turn on or off, or to adjust a brightness. The light 612 provides improved visual conditions such that the camera 610 is configured to obtain image data. The camera 610 and the light 612 facilitate obtaining clear and accurate image data of the interior of a patient's body when the scope 108 is inserted into an opening in the patient's body (e.g., in the patient's spine). The end 608 and the shaft 606 of the scope 108 may have a size (e.g., a diameter) corresponding to an opening produced by a surgeon tool. For example, the surgeon may use an expander that defines an opening into the patient's body through which the scope 108 is configured to be received in order to obtain image data of the interior of the patient's body. The end 608 may have a diameter of approximately 0.25 inches. In some embodiments, the end 608 has a diameter of less than 6 millimeters.

The base portion 602 may include a controller or a processing circuit 614 configured to process any of the data obtained from the camera 610 in the end 608 (e.g., the tip of the scope 108). In some embodiments, wires 604 are coupled with the camera and the light at the end 608 or with the controller and extend from the base portion 602.

The shaft 606 has an elongated and curved shape. In particular, the shaft 606 may have a smaller diameter than the end 608 and also includes a bend 616 at a position along the shaft 606 between the base portion 602 and the end 608. The shaft 606 can have a diameter of 6 millimeters or less such sufficient space or clearance is provided around the shaft 606 for instruments. The bend 616 is defined between a first portion 620 and a second portion 622 of the shaft 606. In some embodiments, the bend 616 is closer to the base portion 602 than the end 608. For example, the first portion 620 may have a shorter length than the second portion 622. The bend 616 may define an angle 618 between a first axis 624 (e.g., a first longitudinal axis, a first centerline, etc.) of the first portion 620 and a second axis 626 (e.g., a second longitudinal axis, a second centerline, etc.) of the second portion 622. In some embodiments, the angle 618 has any value from 35 degrees to 90 degrees between the first portion 620 and the second portion 622 of the shaft 606. The bend 616 can be large enough such that the scope 108 provides space for other disc preparation instruments (e.g., pituitaries, augers, shavers, curettes trials, installers, etc.) to be positioned along side the scope 108 (e.g., along side the second portion 622 of the shaft 606) when using a tube retractor or an endoscope port.

For example, when the end 608 of the scope is inserted into a patient (e.g., into a retractor or other spinal access device), the bend 616 can result in a clearance 632 being defined between the base 602 and the second axis 626 of the second portion 622. The bend 616 is sufficiently large (e.g., a sufficiently large value of the angle 618) such that the clearance 632 provides space for the surgeon to position other disc preparation instruments along side the second portion 632 of the scope 108 without contacting or interfering with the base portion 602. The bend 616 is therefore positioned along the shaft 606 and sized so that the base portion 602 does not obstruct access to a site at which the end 608 is inserted. The scope 108 therefore enables the surgeon to access the site at which the end 608 is inserted with disc preparation instruments without requiring the surgeon to reposition the scope 108. The disc preparation instruments can be inserted in a direction 628 along the second axis 626 or removed in a direction 630 without obstruction from the surgeon's hand that grasps the base portion 602, and without obstruction by the base position 602. Advantageously, the scope 108 provides a structure for the AR display system 100 such that the scope 108 can be used by the surgeon while also using other disc preparation tools during a surgical procedure without requiring repositioning of the end 608 of the scope 108 (and therefore changing the field of view of the camera 610).

A length of the first portion 620 can be varied based on the value of the angle 618. For example, with smaller or shallower angles of the angle 618 (e.g., a 35 degree angle), the length of the first portion 606 (e.g., between the base 602 and the bend 616) may be increased in order to increase the clearance 632. Likewise, if the angle 618 has a larger value (e.g., a 90 degree angle), the length of the first portion 620 may be shorter.

### Configuration of the Exemplary Embodiments

As utilized herein with respect to numerical ranges, the terms “approximately,” “about,” “substantially,” and similar terms generally mean+/−10% of the disclosed values. When the terms “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure.

As used herein, the term “circuit” may include hardware structured to execute the functions described herein. In some embodiments, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some embodiments, a circuit may take the form of one or more analog circuits, electronic circuits (e.g., integrated circuits (IC), discrete circuits, system on a chip (SOCs) circuits, etc.), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

The “circuit” may also include one or more processors communicably coupled to one or more memory or memory devices. In this regard, the one or more processors may execute instructions stored in the memory or may execute instructions otherwise accessible to the one or more processors. In some embodiments, the one or more processors may be embodied in various ways. The one or more processors may be constructed in a manner sufficient to perform at least the operations described herein. In some embodiments, the one or more processors may be shared by multiple circuits (e.g., circuit A and circuit B may comprise or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud based server). To that end, a “circuit” as described herein may include components that are distributed across one or more locations.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

It is important to note that the construction and arrangement of the AR display system 100 as shown in the various exemplary embodiments is illustrative only. Additionally, any element disclosed in one embodiment may be incorporated or utilized with any other embodiment disclosed herein. It should be appreciated that any elements of the various embodiments may be incorporated or utilized with any of the other embodiments disclosed herein.

What is claimed is:

1. An augmented reality (AR) display system for a surgical environment, the AR display system comprising:
a scope comprising a shaft and a camera disposed at an end of the shaft, the shaft including a bend, wherein the end of the shaft is configured to be inserted into a patient;
a wearable AR display device configured to be worn by a surgeon and provide AR imagery to the surgeon; and
processing circuitry configured to:
obtain real-time image data from the scope, X-ray image data of the patient, and navigation data for a surgical procedure of the patient;
generate an AR environment that includes the real-time image data, the X-ray image data, and the navigation data; and
operate the wearable AR display device to provide the AR environment including the real-time image data, the X-ray data, and the navigation data as the AR imagery.

2. The AR display system of claim 1, wherein the processing circuitry is further configured to:
obtain a user input indicating a desired location, size, and orientation of at least one of the real-time image data, the X-ray image data, or the navigation data; and
generate the AR environment according to the desired location, size, and orientation of the at least one of the real-time image data, the X-ray image data, or the navigation data.

3. The AR display system of claim 1, wherein the X-ray image data comprises X-ray imagery of one or more locations of the patient at which the surgical procedure is being performed.

4. The AR display system of claim 1, wherein the navigation data comprises instructions or guidelines for one or more steps for performing the surgical procedure.

5. The AR display system of claim 1, wherein the shaft comprises an elongated tubular member including the camera and a light positioned at the end of the elongated tubular member.

6. The AR display system of claim 5, wherein the bend is defined between a first portion and a second portion of the shaft, the first portion coupled to a base section of the scope, and the second portion defining the end of the shaft, the first portion being shorter than the second portion, wherein the bend is positioned along the shaft and sized to provide clearance between the base section and the second portion such that a site at which the scope is inserted into the patient is accessible by a disc preparation tool without obstruction by the base section.

7. The AR display system of claim 6, wherein the base section configured to provide a surface for the surgeon to hold the scope.

8. The AR display system of claim 1, wherein the scope comprises a wire extending through the shaft from the camera.

9. The AR display system of claim 1, wherein the scope is configured to be inserted into an expander in the patient's body, the scope having an end that is 0.25 inches in diameter or less.

10. A display system for a surgical environment, the display system comprising:
a scope comprising a shaft and a camera disposed at an end of the shaft, the shaft including a bend, wherein the end of the shaft is configured to be inserted into a patient; and
processing circuitry configured to:
obtain real-time image data from the camera of the scope, X-ray image data of the patient from an X-ray database, and navigation data from a navigation database for a surgical procedure of the patient;
generate an AR environment that includes the real-time image data, the X-ray image data, and the navigation data; and
operate a wearable AR display device to provide the AR environment including the real-time image data, the X-ray data, and the navigation data as the AR imagery.

11. The display system of claim 10, wherein the processing circuitry is further configured to:
obtain a user input indicating a desired location, size, and orientation of at least one of the real-time image data, the X-ray image data, or the navigation data; and
generate the AR environment according to the desired location, size, and orientation of the at least one of the real-time image data, the X-ray image data, or the navigation data.

12. The display system of claim 10, wherein the X-ray image data comprises X-ray imagery of one or more locations of the patient at which the surgical procedure is being performed.

13. The display system of claim 10, wherein the navigation data comprises instructions or guidelines for one or more steps for performing the surgical procedure.

14. The display system of claim 10, wherein the shaft comprises an elongated tubular member including the camera and a light positioned at the end of the elongated tubular member.

15. The display system of claim 14, wherein the bend is defined between a first portion and a second portion of the shaft, the first portion coupled to a base section of the scope, and the second portion defining the end of the shaft, the first portion being shorter than the second portion, wherein the bend is positioned along the shaft and sized to provide clearance between the base section and the second portion such that a site at which the scope is inserted into the patient is accessible by a disc preparation tool without obstruction by the base section.

16. The display system of claim 15, wherein the base section configured to provide a surface for the surgeon to hold the scope.

17. The display system of claim 10, wherein the scope comprises a wire extending through the shaft from the camera.

18. A method of providing augmented reality (AR) for a surgical environment, the method comprising:
providing a scope comprising a shaft and a camera disposed at an end of the shaft, the shaft including a bend, wherein the end of the shaft is configured to be inserted into a patient;
obtaining real-time image data from the camera of the scope during a surgical procedure, navigation data for the surgical procedure, and X-ray image data of the patient;
generating an AR environment based on the real-time image data, the navigation data, and the X-ray image data, the AR environment comprising a size and position of the real-time image data, the navigation data, and the X-ray image data; and
operating an AR display device to provide the AR environment comprising the real-time image data, the navigation data, and the X-ray image data.

19. The method of claim 18, further comprising:
obtaining a user input to adjust the size or position of at least one of the real-time image data, the navigation data, or the X-ray image data; and
adjusting the AR environment based on the user input to adjust the size or position of the at least one of the real-time image data, the navigation data, or the X-ray image data.

20. The method of claim 18, wherein the navigation data for the surgical procedure comprises instructions and images of the surgical procedure.
